(19) United States
(12) Patent Application Publication
(10) Pub. No.: US 2025/0032728 A1
(43) Pub. Date: Jan. 30, 2025

(54) Medicament delivery device

(71) Applicant: SHL Medical AG, Zug (CH)

(72) Inventor: Ming-Ting Yin, Taoyuan City (TW)

(21) Appl. No.: 18/716,342

(22) PCT Filed: Nov. 23, 2022
(86) PCT No.: PCT/EP2022/082891

§ 371 Date: Jun. 4, 2024

(30) Foreign Application Priority Data
Dec. 7, 2021 (EP) 21212699.9

(51) Int. Cl. A61M 5/32; A61M 5/315

(52) U.S. Cl. CPC A61M 5/3204; A61M 5/3157; A61M 2205/582

(57) ABSTRACT

The present disclosure provides a medicament delivery device including a housing having a proximal housing end and a distal housing end, a user-operated component permanently attached to the housing, and arranged to move from a first position to a second position relative to the housing, a delivery member cover protruding from the proximal housing end, and configured to be moved from an extended position relative to the housing to a retracted position, and a blocking sleeve arranged axially fixed relative to the housing when the user-operated component is in the first position. The blocking sleeve has a proximally facing surface axially aligned with and facing a distally facing surface of the delivery member cover when the user-operated component is in the first position, preventing the delivery member cover from moving to the retracted position. The user-operated component is configured to move the proximally facing surface to a position offset from the distally facing surface when the user-operated component is moved to the second position, allowing the delivery member cover to move from the extended position to the retracted position.

### CROSS-REFERENCE TO RELATED APPLICATIONS

The present application is a U.S. National Phase Application pursuant to 35 U.S.C. § 371 of International Application No. PCT/EP2022/082891 filed Nov. 23, 2022, which claims priority to European Patent Application No. 21212699.9 filed Dec. 7, 2021. The entire disclosure contents of these applications are herewith incorporated by reference into the present application.

### TECHNICAL FIELD

The present disclosure generally relates to medicament delivery devices.

### BACKGROUND

Medicament delivery devices such as autoinjectors may have a linearly movable needle cover extending from the housing. The needle is exposed when the needle cover is pushed into the housing. In some cases, the needle cover may also trigger medicament delivery.

There are medicament delivery devices that have a removable rear cap provided with a safety clip that extends radially into the housing and prevents the needle cover to move in the distal direction of the device, i.e., towards the rear end of the device. When the rear cap with its safety clip has been removed, needle cover is able to move further into the housing.

### SUMMARY

An object of the present disclosure is to provide a medicament delivery device which solves, or at least mitigates, problems of the prior art.

There is hence provided a medicament delivery device comprising: a housing having a proximal housing end and a distal housing end, a user-operated component permanently attached to the housing, and arranged to move from a first position to a second position relative to the housing, a delivery member cover protruding from the proximal housing end, and configured to be moved from an extended position relative to the housing to a retracted position, a blocking sleeve arranged axially fixed relative to the housing when the user-operated component is in the first position, wherein the blocking sleeve has a proximally facing surface axially aligned with and facing a distally facing surface of the delivery member cover when the user-operated component is in the first position, preventing the delivery member cover from moving to the retracted position, wherein the user-operated component is configured to move the proximally facing surface to a position offset from the distally facing surface when the user-operated component is moved to the second position, allowing the delivery member cover to move from the extended position to the retracted position.

The user-operated component, which is irremovably attached to the housing, is configured to set the delivery member cover free to move from the extended position to the retracted position when in the second position. The user-operated component is thus not removed from the housing when moved from the first position to the second position.

Since the user-operated component is permanently attached to the housing, the user-operated component can easily be reset to the first position without the risk that it becomes lost.

Further, since the user-operated component is permanently attached to the housing, it will not come loose in the event of a shock, for example in case the medicament delivery device is dropped.

The proximally facing surface is a radial surface.

The distally facing surface is a radial surface.

The medicament delivery device may be an autoinjector.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device.

When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component.

Similarly, “radial” or “radially” refer to a direction extending radially relative to the axis, and “rotation”, “rotational” and “rotationally” refer to rotation relative to the axis.

According to one embodiment the blocking sleeve is an outer sleeve and the user-operated component is a knob, wherein the first position is a first rotational position, the second position is a second rotational position, and the offset is a circumferential offset, wherein the knob is configured to rotationally engage with the outer sleeve such that the outer sleeve is rotated with the knob to the second position.

According to one embodiment the housing has a radially inwards extending blocking structure which when the knob is in the first position is arranged distally relative to a distally directed face of the outer sleeve, preventing movement of the outer sleeve towards the distal housing end.

According to one embodiment the knob is configured to rotate the outer sleeve with such an amount to reach the second position that the distally directed face becomes circumferentially fully offset from the blocking structure.

According to one embodiment the outer sleeve comprises an axial slot extending in a direction towards the proximal housing end from the distally directed face, the axial slot being configured to receive the blocking structure when the knob is in the second position, allowing the outer sleeve to move towards the distal housing end caused by movement of the delivery member cover.

According to one embodiment the blocking sleeve is an inner sleeve, wherein the user-operated component is a slidable switch, wherein the offset is a radial offset.

According to one embodiment the first position is a first axial position, and the second position is a second axial position in which the slidable switch is closer to the proximal housing end than in the first position.

According to one embodiment the inner sleeve comprises a flexible inner sleeve tab axially aligned with the slidable switch and provided with the proximally facing surface, wherein the inner sleeve tab in a default state extends radially outwards with the proximally facing surface facing the distally facing surface, wherein in the second position the slidable switch is configured to press the inner sleeve tab inwards radially below the delivery member cover so that the proximally facing surface is fully offset from the distally facing surface.

According to one embodiment the delivery member cover comprises a radially flexible radially outwards extending delivery member tab arranged radially outwards of the inner sleeve tab, wherein the slidable switch is configured to press the delivery member tab onto the inner sleeve tab to press the inner sleeve tab radially below the delivery member cover.

One embodiment comprises an outer sleeve arranged radially outside of the inner sleeve, the outer sleeve having an outer sleeve arm configured to engage with the inner sleeve, wherein in the first position the slidable switch is arranged radially outside the outer sleeve arm, preventing the outer sleeve arm to flex out radially and disengage from the inner sleeve.

According to one embodiment the inner sleeve has a recess, wherein the outer sleeve arm is configured to engage with the recess.

One embodiment comprises: a plunger rod, an inner sleeve arranged axially fixed relative to the housing, and arranged radially outside the plunger rod, the inner sleeve having radially flexible arms configured to engage with the plunger rod to hold the plunger rod in an initial position, an outer sleeve arranged radially outside the inner sleeve, the outer sleeve having a radial inner dimension which prevents the arms from flexing out radially when the delivery member cover is in the extended position, wherein the blocking sleeve is one of the inner sleeve and the outer sleeve, and wherein the delivery member cover is configured to move the outer sleeve relative to the inner sleeve, in a direction towards the distal housing end, when the delivery member cover is moved towards the retracted position, the arms thereby being able to flex out radially and disengage from the plunger rod.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of a first example of a medicament delivery device;

FIG. 2 is an exploded view of the medicament delivery device in FIG. 1;

FIG. 3 is a rear perspective view of the medicament delivery device in FIG. 1, with the housing shown transparently, when the user-operated is in its first position;

FIG. 4 shows the view in FIG. 3 with the housing removed;

FIG. 5 is a longitudinal section of a rear portion of the medicament delivery device in FIG. 1 in an default state;

FIG. 6 is a rear perspective view of the medicament delivery device in FIG. 1, with the housing shown transparently when the user-operated component has been set in its second position;

FIG. 7 shows the view in FIG. 6 with the housing removed;

FIG. 8 is a longitudinal section of a rear portion of the medicament delivery device in FIG. 1 when the user-operated component has been set in the second position and the plunger rod is about to be released;

FIG. 9 is a perspective view of a second example of a medicament delivery device;

FIG. 10 shows various components of the medicament delivery device in FIG. 9;

FIG. 11 is a longitudinal section of a rear portion of the medicament delivery device in FIG. 9 in an default state; and

FIG. 12 is a longitudinal section of a rear portion of the medicament delivery device in FIG. 9 when the user-operated component has been set in the second position but before the delivery member cover has been moved to the retracted position.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

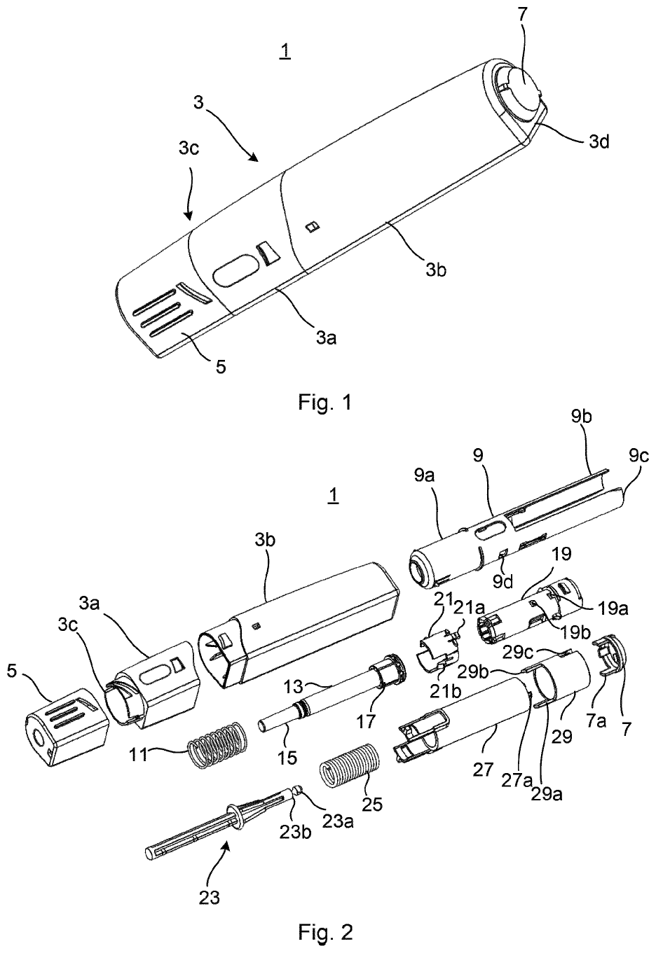

FIG. 1 shows a first example of a medicament delivery device 1. The medicament delivery device 1 is an auto-injector but could alternatively for example be a jet injector.

The medicament delivery device 1 comprises a housing 3. The exemplified housing 3 comprises a front shell 3a and a rear shell 3b. The front shell 3a is connected to the rear shell 3b. The connection may for example be a snap fit connection. Alternatively, the housing 3 may consist of a single integral component.

The medicament delivery device 1 comprises a cap 5 configured to be detachably attached to the housing 3. In the example, the cap 5 is configured to be attached to the front shell 3a.

The housing 3 has a proximal housing end 3c, which in FIG. 1 is covered by the cap 5, and a distal housing end 3d.

The medicament delivery device 1 comprises a user-operated component 7 permanently attached to the housing 3. The permanent attachment may for example be by means of a snap fit connection.

In the present example, the user-operated component 7 is a knob. The knob is provided at the distal housing end 3d.

The knob is movable from a first position relative to the housing 3 to a second position. The first position is a first rotational position, and the second position is a second rotational position. In the present example, the knob is configured to rotate about a longitudinal axis of the housing 3, or about a longitudinal axis parallel with the longitudinal axis of the housing 3.

FIG. 2 is an exploded view of the medicament delivery device 1.

The medicament delivery device 1 comprises a delivery member cover 9. The delivery member cover 9 protrudes from the proximal housing end 3c.

The delivery member cover 9 is covered by the cap 5 when the cap 5 is attached to the housing 3.

The delivery member cover 9 comprises a tubular proximal portion 9a and distally extending legs 9b extending from the tubular proximal portion 9a.

The delivery member cover 9 is movably arranged in the housing 3. The delivery member cover 9 is configured to be moved from an extended position to a retracted position relative to the housing 3. The delivery member cover 9 received further by the housing 3 in the retracted position compared to the extended position.

The delivery member cover 9 can be moved from the extended position to the retracted position by pushing the medicament delivery device 1 against an injection site with the delivery member cover 9 directed towards the injection site.

The delivery member cover 9 is biased towards the extended position.

The medicament delivery device comprises a first resilient member 11 configured to bias the delivery member cover 9 towards the extended position. The first resilient member 11 may be a spring such as a coil spring.

The delivery member cover 9 is configured to return to the extended position from the retracted position when the force applied by the first resilient member 11 onto the delivery member cover 9 overcomes that applied onto the delivery member cover 9 by a user.

A medicament container 13 such as a syringe may be arranged in the medicament delivery device 1. The medicament container 13 may comprise a delivery member such as a needle. Further, a delivery member shield 15 may be provided around the delivery member, e.g., the needle. The delivery member shield 15 may comprise a flexible needle shield and/or a rigid needle shield.

The exemplified medicament delivery device 1 comprises a medicament container support sleeve 17 arranged around a distal end portion of the medicament container 13. The medicament container support sleeve 17 may be arranged proximally of and adjacent to an end flange of the medicament container 13.

The medicament delivery device 1 comprises a medicament container holder 19. The medicament container holder 19 is axially fixed relative to the housing 3.

The medicament container support sleeve 17 is configured to be received by the medicament container holder 19 to maintain the medicament container 13 in an axially fixed relation with the medicament container holder 19. The medicament container holder 19 may be provided with an inner distally facing radial ledge configured to support a proximal end of the medicament container support sleeve 17.

The exemplified medicament delivery device 1 comprises a lock out collar 21. The lock out collar 21 is configured to be arranged radially outside the medicament container holder 19. The lock out collar 21 is configured to move axially relative to the medicament container holder 19, from an initial distal position when the delivery member cover 9 initially is in the extended position to a final proximal position when the delivery member cover 9 has returned to the extended position from the retracted position.

The lock out collar 21 has a grabbing arm 21a extending radially inwards. The grabbing arm 21a is configured to engage with a distal opening 19a of the medicament container holder 19 in the initial distal position when the delivery member cover 9 is initially in the extended position.

The lock out collar 21 has a radially outwards extending structure 21b arranged proximally axially offset from a radial opening 9d of the delivery member cover 9 when the delivery member cover 9 is in the extended position. The radial opening 9d is configured to receive the radially outwards extending structure 21b when the delivery member cover 9 is in the retracted position.

When the delivery member cover 9 is moved distally towards the retracted position, it moves relative to the lock out collar 21 to a position in which the radially outwards extending structure 21b radially aligns with and engages with the radial opening 9d. When the delivery member cover 9 is returned to the extended position, it brings with it the lock out collar 21. The grabbing arm 21a thus disengages from the distal opening 19a. When the delivery member cover 9 reaches the extended position the grabbing arm 21a engages with a proximal opening 19b of the medicament container holder 19, axially aligned with the distal opening 19a. The grabbing arm 21 has a distally facing radial surface and the proximal opening 19b has a proximally facing radial surface which faces the distally facing radial surface when the grabbing arm 21a has engaged with the proximal opening 19b. Thus, when the delivery member cover 9 has returned to the extended position, it is prevented from moving to the retracted position.

The medicament delivery device 1 comprises a plunger rod 23. The plunger rod 23 has a distal plunger rod end provided with a bud structure 23a.

The plunger rod 23 is configured to extend into the medicament container 13.

The plunger rod 23 is biased in the proximal direction.

The medicament delivery device 1 comprises a second resilient member 25. The second resilient member 25 may be a spring such as a coil spring. The second resilient member 25 is configured to bias the plunger rod 23 in the proximal direction.

The medicament delivery device 1 comprises an inner sleeve 27. The inner sleeve 27 is configured to engage with the housing 3. The inner sleeve 27 is thus axially fixed relative to the housing 3. The inner sleeve 27 may for example be configured to engage with the housing 3 by means of a snap fit connection, or radial arms.

The inner sleeve 27 has radially flexible arms 27a configured to engage with the plunger rod 23 to hold the plunger rod 23 in an initial position. The radially flexible arms 27a are configured to extend towards each other in a narrowed down region between the bud structure 23a and a distal shoulder 23b of the plunger rod 23, from which the bud structure 23a extends.

The medicament delivery device 1 comprises a blocking sleeve 29. In this example the blocking sleeve 29 is an outer sleeve and will in this example in the following be referred to as “outer sleeve 29”.

The outer sleeve 29 is arranged concentrically with and radially outside the inner sleeve 27.

The outer sleeve 29 has a radial inner dimension which prevents the radially flexible arms 27a from flexing out radially when the delivery member cover 9 is in the extended position. The outer sleeve 29 bears against the radially flexible arms 27a when the delivery member cover 9 is in the extended position. The plunger rod 23 is thus held in the initial position by the inner sleeve 27.

The outer sleeve 29 comprises proximally extending legs 29a. Each proximally extending leg 29a has a proximally facing surface 29b axially aligned with and facing a distally facing surface 9c of a respective one of the distally directed legs 9b of the delivery member cover 9 when the delivery member cover 9 is in the extended position. Each proximally facing surface 29b may be abutting a distally facing surface 9c when the delivery member cover 9 is in the extended position.

The outer sleeve 29 is configured to be rotationally engaged with the user-operated component 7, i.e., the knob. The outer sleeve 29 is thus rotatable concurrently with the user-operated component 7 when the user-operated component 7 is rotated relative to the housing 3.

The outer sleeve 29 has a distal axial slot 29c configured to engage with a proximally extending protrusion 7a of the user-operated component 7. The outer sleeve 29 is rotationally fixed to the user-operated component 7 by means of the connection between the distal axial slot 29c and the proximally extending protrusion 7a received by the distal axial slot 29c.

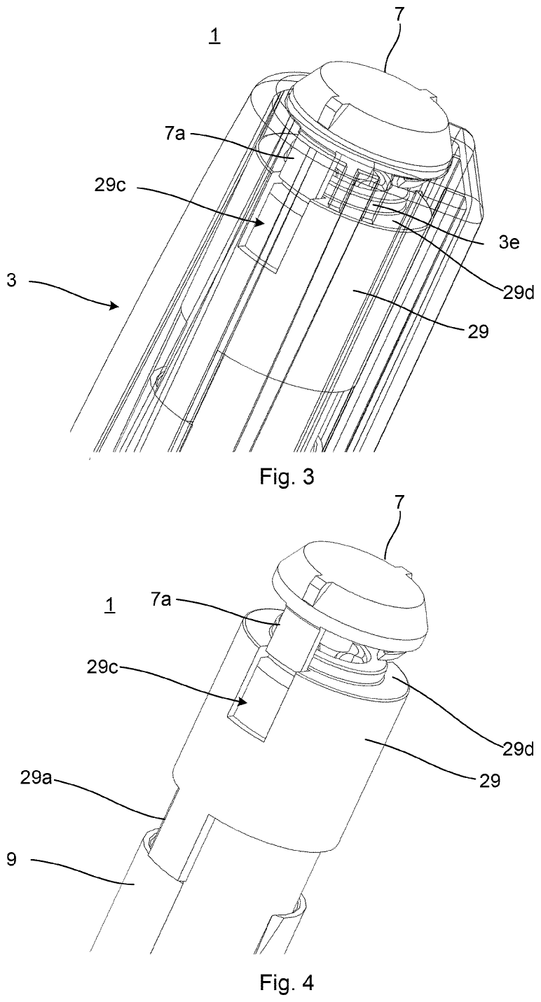

FIG. 3 shows a rear portion of the medicament delivery device 1 with the housing 3 made transparent when the delivery member cover 9 is in the extended position and the user-operated component 7 is in the first position. The proximally extending protrusion 7a of the user-operated component 7 engages with the distal axial slot 29c of the outer sleeve 29.

The housing 3 has a radially inwards extending blocking structure 3e which is arranged distally of and bears against a distally directed face 29d of the outer sleeve 29 when the delivery member cover 9 is in the extended position and the user-operated component 7 is in the first position. The delivery member cover 9 is thus prevented from moving towards the retracted position.

FIG. 4 shows a rear portion of the medicament delivery device 1 with the housing 3 removed in the same state as shown in FIG. 3.

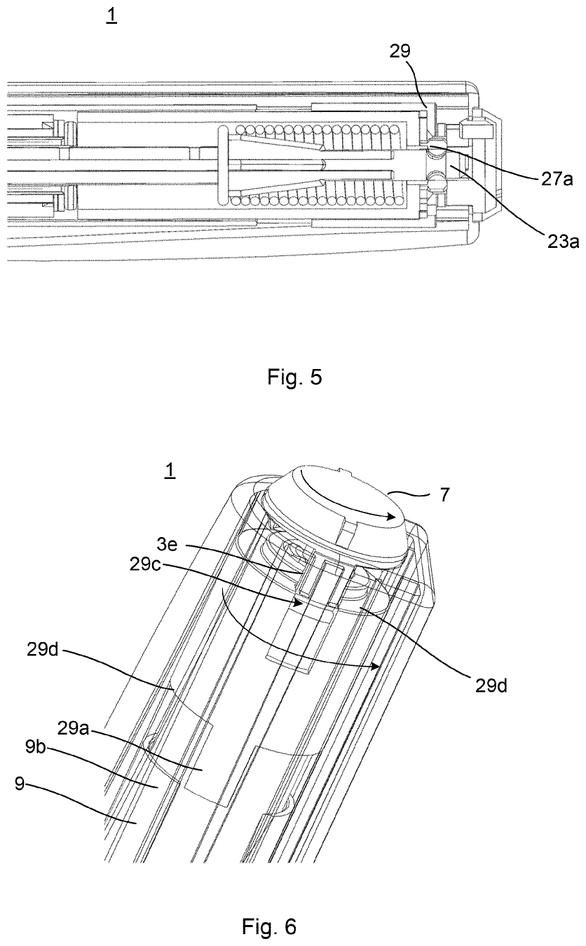

FIG. 5 shows a rear portion of the medicament delivery device 1, in a longitudinal section along a longitudinal axis of the medicament delivery device 1.

The radially flexible arms 27a engage with the plunger rod 23a in the narrowed down region located proximally of the bud structure 23a. The outer sleeve 29 has a collar portion arranged radially outside the radially flexible arms 27a. The collar portion has a radial inner dimension that prevents the radially flexible arms 27a to flex radially outwards when the outer sleeve 29 is in its initial outer sleeve position.

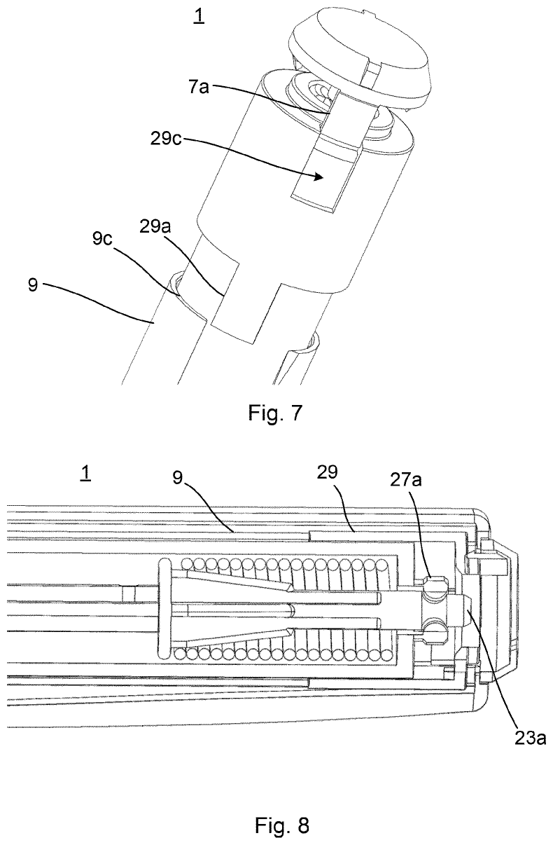

FIGS. 6-7 show the medicament delivery device 1 when the user-operated component 7 has been rotated from the first position to the second position. Since the user-operated component 7 is rotationally engaged with the outer sleeve 29, the outer sleeve 29 is rotated together with the user-operated component 7.

The proximally extending legs 29a are arranged circumferentially fully offset from the distally extending legs 9b of the delivery member cover 9. Thus, the proximally facing surface 29a of the outer sleeve 29 is fully circumferentially offset from the distally facing surface 9c of the delivery member cover 9.

The blocking structure 3e of the housing 3 has become axially aligned with the distal axial slot 29c. The distal axial slot 29c is designed to be able to receive the blocking structure 3e. The outer sleeve 29 is thus set free to move axially towards the distal housing end 3d.

The proximally extending legs 29a of the delivery member cover 9 are axially spaced apart from a proximal edge 29d of the outer sleeve 29, from which the proximally extending legs 29a extend when the delivery member cover 9 is in the extended position. The delivery member cover 9 is thus initially able to move in the proximal direction towards the retracted position without moving the outer sleeve 29. Movement of the delivery member cover 9 towards the retracted position and the outer sleeve 29 eventually results in that the distally facing surface 9c contacts the proximal edge 29d. Further distal movement of the delivery member cover 9 causes movement of the outer sleeve 29 towards the distal housing end 3d, as shown in FIG. 8.

As shown in FIG. 8, the radially flexible arms 27a are allowed to flex radially outwards when the outer sleeve 29 has been moved axially towards the distal housing end 3d because the collar portion has been moved distally from the radially flexible arms 27a.

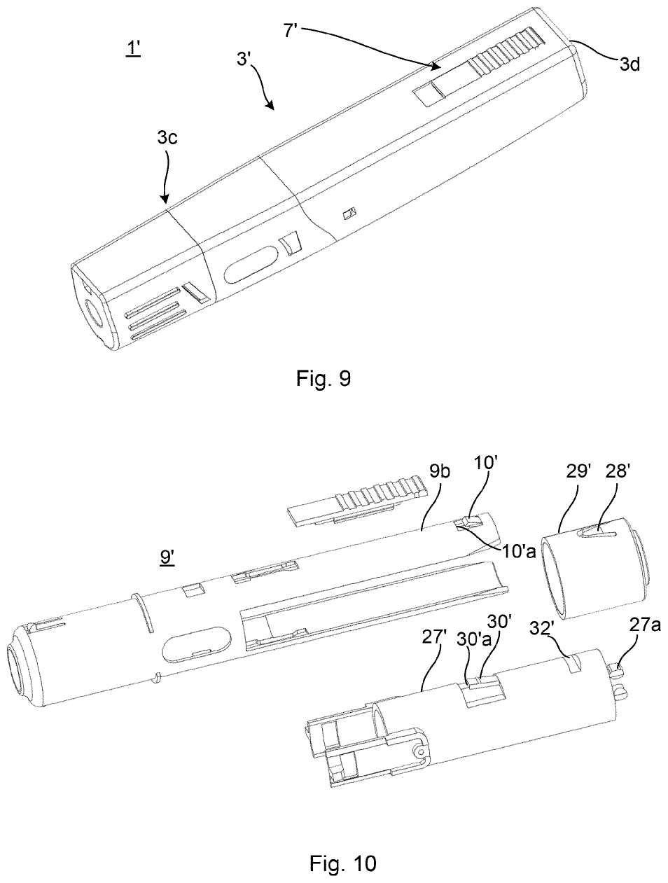

FIG. 9 shows a second example of a medicament delivery device. The medicament delivery device 1′ is an auto-injector but could alternatively for example be a jet injector.

The medicament delivery device 1′ is similar to the medicament delivery device 1, except for the mechanism that involves the user-operated component and the initial blocking of the delivery member cover in the extended position.

The medicament delivery device 1′ comprises a user-operated component 7′ which is a slidable switch. The user-operated component 7′ is configured to move parallel with a longitudinal axis of the medicament delivery device 1′ from a first position to a second position relative to the housing 3′. The first position is a first axial position, and the second position is a second axial position. The slidable switch 7′ is closer to the proximal housing end 3c when the user operated component 7′ is in the second position. The user-operated component 7′ is shown in the first position in FIG. 9.

The user-operated component 7′ is permanently attached to the housing 3′.

FIG. 10 shows components of the medicament delivery device 1′ that differ from the corresponding components of the medicament delivery device 1 of the first example.

The delivery member cover 9′ of the second example in addition to the structure described in the first example, comprises a radially flexible radially outwards extending delivery member tab 10′. The delivery member tab 10′ is arranged at a proximal end portion of one of the distally extending legs 9b of the delivery member cover 9′.

The delivery member tab 10′ has a ramp-like shape that decreases radially in a direction towards the distal housing end 3d.

The delivery member cover 9′ has a distally facing surface 10′a. The distally facing surface 10′a is arranged in an opening in which the delivery member tab 10′ extends axially towards the distally facing surface 10′a.

The inner sleeve 27′ is in this example the blocking sleeve. The inner sleeve 27′ is arranged radially inside of the delivery member cover 9′.

The inner sleeve 27′ has a flexible inner sleeve tab 30′. The flexible inner sleeve tab 30′ extends radially outwards. The flexible inner sleeve tab 30′ is radially flexible. The flexible inner sleeve tab 30′ has a ramp-like shape that decreases radially in a direction towards the distal housing end 3d.

The flexible inner sleeve tab 30′ has a proximally facing surface 30′a. When the delivery member cover 9′ is in the extended position initially, i.e., when the medicament delivery device 1′ is in a default state, the flexible inner sleeve tab 30′ extends radially outwards into the opening and the proximally facing surface 30′a faces the distally facing surface 10′a. The proximally facing surface 30′a thus blocks the delivery member cover from moving towards the retracted position.

When the user-operated component 7′ is moved to the second position, i.e., in the proximal direction, the user-operated component 7′ is configured to press the delivery member tab 10′ radially inwards onto the flexible inner sleeve tab 30′. The user-operated component 7′ has such a radial thickness that it bears against and presses the delivery member tab 10′ radially inwards when moved to the second position. The flexible inner sleeve tab 30′ is pressed inwards radially below the delivery member cover 9′ so that the proximally facing surface 30′a is fully offset radially from the distally facing surface 10′a.

The outer sleeve 29′ has an outer sleeve arm 28′ configured to engage with the inner sleeve 27′. The inner sleeve 27′ has a recess 32′ configured to receive the outer sleeve arm 28′. The outer sleeve 29′ thereby engages with the inner sleeve 27′.

In the first position, the user-operated component 7′ is arranged radially outside the outer sleeve arm 28′. The user-operated component 7′ is in the first position configured to press the outer sleeve arm 28′ radially inwards into the recess 32′. The outer sleeve arm 28′ is thus not able to flex radially outwards and disengage from the inner sleeve 29′. The outer sleeve 29′ is therefore axially fixed relative to the inner sleeve 29′ when the user-operated component 7′ is in the first position. This reduces the risk of movement of the outer sleeve 29 if the medicament delivery device 1′ is accidentally dropped for example.

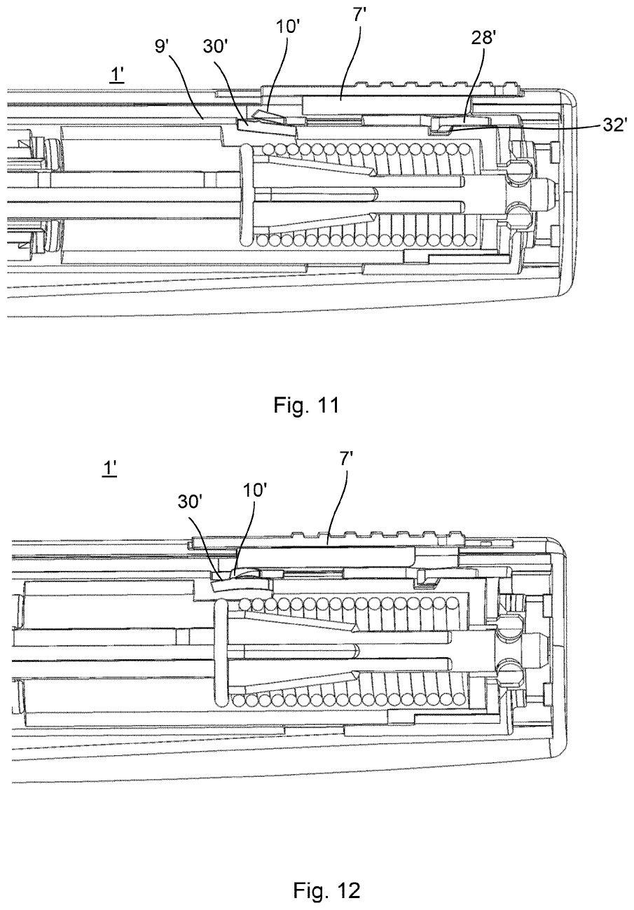

FIG. 11 shows a longitudinal section of a rear portion of the medicament delivery device 1′ in its default state, i.e., before the delivery member cover 9′ has been moved from the extended position to the retracted position. The outer sleeve arm 28′ is engaged with the recess 32′, preventing the outer sleeve 29′ to move towards the distal housing end 3d.

The flexible inner sleeve tab 30′ extends radially outwards into the opening and the proximally facing surface 30′a faces the distally facing surface 10′a, preventing the delivery member cover 9 from moving towards the retracted position.

FIG. 12 shows the medicament delivery device 1′ when the user-operated component 7′ is in the second position, before the delivery member cover 9′ has been moved to the retracted position.

The user-operated component 7′ presses down the delivery member tab 10′ over the flexible inner sleeve tab 30′ which thus flexes radially inwards to a level below the inner surface of the delivery member cover 9′. Moreover, the outer sleeve arm 28 is set free to flex radially outwards due to the displacement of the user-operated component 7′. The delivery member cover 9′ is thus allowed to move from the extended position to the retracted position. Initial movement of the delivery member cover 9′ will not cause movement of the outer sleeve 29′. Eventually the delivery member cover 9′ will reach the outer sleeve 29′, causing the outer sleeve 29′ to move towards the distal housing end 3d as the delivery member cover 9′ is moved further distally. The radially flexible arms 27a of the inner sleeve 27 will thus be able to flex out and release the plunger rod 23.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the drug delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the drug delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

1-12. (canceled)

13. A medicament delivery device comprising:
a housing having a proximal housing end and a distal housing end;
a user-operated component permanently attached to the housing, and arranged to move from a first position to a second position relative to the housing;
a delivery member cover protruding from the proximal housing end, and configured to be moved from an extended position relative to the housing to a retracted position; and
a blocking sleeve arranged axially fixed relative to the housing when the user-operated component is in the first position,
wherein the blocking sleeve has a proximally facing surface axially aligned with and facing a distally facing surface of the delivery member cover when the user-operated component is in the first position, preventing the delivery member cover from moving to the retracted position,
wherein the user-operated component is configured to move the proximally facing surface to a position offset from the distally facing surface when the user-operated component is moved to the second position, allowing the delivery member cover to move from the extended position to the retracted position.

14. The medicament delivery device of claim 13, wherein the blocking sleeve is an outer sleeve and the user-operated component is a knob, wherein the first position is a first rotational position, the second position is a second rotational position, and the offset is a circumferential offset, wherein the knob is configured to rotationally engage with the outer sleeve such that the outer sleeve is rotated with the knob to the second position.

15. The medicament delivery device of claim 14, wherein the housing has a radially inwards extending blocking structure which when the knob is in the first position is arranged distally relative to a distally directed face of the outer sleeve, preventing movement of the outer sleeve towards the distal housing end.

16. The medicament delivery device of claim 15, wherein the knob is configured to rotate the outer sleeve with such an amount to reach the second position that the distally directed face becomes circumferentially fully offset from the blocking structure.

17. The medicament delivery device of claim 15, wherein the outer sleeve comprises an axial slot extending in a direction towards the proximal housing end from the distally directed face, the axial slot being configured to receive the blocking structure when the knob is in the second position, allowing the outer sleeve to move towards the distal housing end caused by movement of the delivery member cover.

18. The medicament delivery device of claim 13, wherein the blocking sleeve is an inner sleeve, wherein the user-operated component is a slidable switch, wherein the offset is a radial offset.

19. The medicament delivery device of claim 18, wherein the first position is a first axial position, and the second position is a second axial position in which the slidable switch is closer to the proximal housing end than in the first position.

20. The medicament delivery device of claim 18, wherein the inner sleeve comprises a flexible inner sleeve tab axially aligned with the slidable switch and provided with the proximally facing surface, wherein the inner sleeve tab in a default state extends radially outwards with the proximally facing surface facing the distally facing surface, wherein in the second position the slidable switch is configured to press the inner sleeve tab inwards radially below the delivery member cover so that the proximally facing surface is fully offset from the distally facing surface.

21. The medicament delivery device of claim 20, wherein the delivery member cover comprises a radially flexible radially outwards extending delivery member tab arranged radially outwards of the inner sleeve tab, wherein the slidable switch is configured to press the delivery member tab onto the inner sleeve tab to press the inner sleeve tab radially below the delivery member cover.

22. The medicament delivery device of claim 18, comprising an outer sleeve arranged radially outside of the inner sleeve, the outer sleeve having an outer sleeve arm configured to engage with the inner sleeve, wherein in the first position the slidable switch is arranged radially outside the outer sleeve arm, preventing the outer sleeve arm to flex out radially and disengage from the inner sleeve.

23. The medicament delivery device of claim 22, wherein the inner sleeve has a recess, and wherein the outer sleeve arm is configured to engage with the recess.

24. The medicament delivery device of claim 13, further comprising:
a plunger rod;
an inner sleeve arranged axially fixed relative to the housing, and arranged radially outside the plunger rod, the inner sleeve having radially flexible arms configured to engage with the plunger rod to hold the plunger rod in an initial position; and
an outer sleeve arranged radially outside the inner sleeve, the outer sleeve having a radial inner dimension which prevents the arms from flexing out radially when the delivery member cover is in the extended position,
wherein the blocking sleeve is one of the inner sleeve and the outer sleeve, and
wherein the delivery member cover is configured to move the outer sleeve relative to the inner sleeve, in a direction towards the distal housing end, when the delivery member cover is moved towards the retracted position, the arms thereby being able to flex out radially and disengage from the plunger rod.
